(19) United States
(12) Patent Application Publication
(10) Pub. No.: US 2015/0290306 A1
(43) Pub. Date: Oct. 15, 2015

(54) COMPOSITIONS AND METHODS FOR DIAGNOSIS AND TREATMENT OF MALIGNANT GLIOMAS

(71) Applicant: David E. ANDERSON, (US)

(72) Inventor: David E. Anderson, Boston, MA (US)

(21) Appl. No.: 14/439,189

(22) PCT Filed: Oct. 28, 2013
(86) PCT No.: PCT/US13/67081

§ 371 Date: Apr. 28, 2015

Related U.S. Application Data
(60) Provisional application No. 61/719,681, filed on Oct. 29, 2012.

(51) Int. Cl. A61K 39/00; G01N 33/68

(52) U.S. Cl. CPC A61K 39/0011; G01N 33/6866; G01N 33/6869; A61K 2039/585; A61K 2039/552; A61K 2039/55505; A61K 2039/55555; A61K 2039/55572; A61K 2039/55577; A61K 2039/55511; A61K 2039/55561; G01N 2333/57; G01N 2333/5409; G01N 2800/52

(57) ABSTRACT

The present application provides compositions and methods useful for the diagnosing and treating malignant gliomas. As described herein, the compositions and methods are based on the development of HLA class II binding peptides and peptide antigens encoded by the MAGE-A3 and IL-13Rα2 tumor associated genes, which stimulate the activity and proliferation of CD4+ T lymphocytes. In embodiments described herein, the compositions may induce a therapeutic response against malignant gliomas.

### CROSS REFERENCE TO RELATED APPLICATION

This application claims priority to and benefit of U.S. Provisional Patent Application Ser. No. 61/719,681 filed Oct. 29, 2012, which is hereby incorporated herein by reference in its entirety.

### FIELD

This disclosure relates to fragments of the glioma-associated antigens MAGE and IL-13 receptor α2. The peptides and compositions of the peptides are useful in therapeutic and diagnostic contexts.

### SEQUENCE LISTING

The instant application contains a Sequence Listing which has been submitted in ASCII format via EFS-Web and is hereby incorporated by reference in its entirety. Said ASCII copy, created on Sep. 26, 2013, is named ANDEDAV-3-35608_SL.txt and is 3,574 bytes in size.

### BACKGROUND/INTRODUCTION

Brain cancer is the leading cause of cancer-related death in patients younger than age 35 and accounts for roughly 10% of all cancers diagnosed in North America. Treatment of brain tumours is complicated by the fact that there are more than 120 different types, which range from low grade astrocytomas to high grade glioblastomas (GBM). Malignant gliomas such as GBM are by far the most common brain cancer found in adults and one of the most difficult to treat. Even with aggressive single and multimodal treatment options such as surgery, chemotherapy, radiation and small molecule inhibitors, the survival has remained unchanged over the past three decades with a median survival of less than one year after diagnosis. Reasons for the failure of conventional treatments is multifactorial including the highly infiltrative/invasive nature of GBM, limitation of drug delivery through the blood brain barrier and neural parenchyma, and genetic heterogeneity resulting in intrinsic resistance to available treatments and the rise of aggressive resistant clones. Therefore, there is a dire requirement for new treatment options for GBM.

Vaccination against tumor-associated antigens is one promising approach to immunotherapy against malignant gliomas. While previous vaccine efforts have focused exclusively on HLA class I-restricted peptides, class II-restricted peptides are necessary to induce CD4⁺ helper T cells and sustain effective anti-tumor immunity. The investigation described herein assessed the ability of five candidate peptide epitopes derived from glioma-associated antigens MAGE and IL-13 receptor α2 to detect and characterize CD4⁺ helper T cell responses in the peripheral blood of patients with malignant gliomas.

Therapeutic vaccine strategies to shift tumor antigen-specific T cell response to a more immunostimulatory Th1 bias may be needed for immunotherapeutic trials to be more successful clinically.

### SUMMARY

The present application provides compositions and methods useful for diagnosing and treating malignant gliomas. As described herein, the compositions and methods are based on the development of HLA class II binding peptides and peptide antigens encoded by the MAGE-A3 and IL-13 receptor α2 tumor associated genes, which stimulate the activity and proliferation of CD4+ T lymphocytes.

There is provided herein immunogenic compositions that include combinations of peptides. It is to be understood that the following exemplary combinations are non-limiting and that the present application encompasses all permutations and combinations of the peptides described herein. In certain embodiments, each peptide in an immunogenic composition is independently immunogenic. In some embodiments, the immunogenic compositions comprise one or more peptides comprising a region having at least 75%, 80%, 85%, 90% or 95% sequence identity with 16-49 contiguous amino acids of SEQ ID NO. 1, wherein the one or more peptides comprise 49 or fewer contiguous amino acids from MAGE A3 protein. In some embodiments, the immunogenic compositions comprise one or more peptides comprising a region having at least 75%, 80%, 85%, 90% or 95% sequence identity with 16-25 contiguous amino acids of SEQ ID NO. 2, wherein the one or more peptides comprise 25 or fewer contiguous amino acids from IL-13Rα2 protein. In some embodiments, the immunogenic compositions comprise one or more peptides comprising a region having at least 75%, 80%, 85%, 90% or 95% sequence identity with 16-49 contiguous amino acids of SEQ ID NO. 1, wherein the one or more peptides comprise 49 or fewer contiguous amino acids from MAGE A3 protein and one or more peptides comprising a region having at least 75%, 80%, 85%, 90% or 95% sequence identity with 16-25 contiguous amino acids of SEQ ID NO. 2, wherein the one or more peptides comprise 25 or fewer contiguous amino acids from IL-13Rα2 protein.

Both primary and recurrent GBM patients are unlikely to have the capacity to favorably respond to immunization against tumor antigens that involve peptide and subunit vaccines with weak ability to promote Th1 immunity. Indeed, suboptimal vaccination could even enhance the immunosuppressive status of patients, as recently demonstrated when HLA class II-restricted peptide vaccination induced regulatory T cells with potential to exacerbate the immunosuppressive state in the patients (Francois V., et al: Cancer Res 2009, 69(10):4335-4345. In another recent clinical trial conducted in melanoma patients that involved multiple HLA class II-restricted peptides from MAGE and melanocytic differentiation antigen, vaccine-induced T helper cell responses were induced in a majority of the patients (81%), yet beneficial clinical responses were observed in only two out of 17 patients (Slingluff C L, et al: J Clin Oncol 2008, 26(30):4973-4980).

Studies have demonstrated that resection of tumor or achievement of disease free status can restore Th1 immunity in patients with malignant diseases such as malignant melanomas and renal cell carcinomas (Nevala W K, et al: Clin Cancer Res 2009, 15(6):1931-1939; Tatsumi T, et al: J Exp Med 2002, 196(5):619-628). Successful resection of gliomas may reverse an unfavorable background that promotes Th2 bias in these patients, and may represent an ideal time at which to administer a therapeutic vaccine, such as the immunogenic composition described herein.

As described herein the formulation of GBM peptide antigens with TLR agonists, in particular the TLR9 agonist CpG, can be used to further reverse the Th2 bias directed against these antigens as well as ameliorate the suppressive activity associated with regulatory T cells directed against the same antigens. Further information regarding such suppressive activity can be found in the following references: (Jacobs C, et al: Int J Cancer, 128(4):897-907; LaRosa D F, et al: Immunol Lett 2007, 108(2):183-188; Peng G, et al: Science 2005, 309(5739):1380-1384; Sharma M D, et al: Immunity, 33(6):942-954; Urry Z, et al: J Clin Invest 2009, 119(2):387-398).

Moreover, the antigens defined herein are applicable to diagnosis and vaccination of patients with melanoma, given that MAGE antigens are frequently over-expressed among melanomas, and functional evidence of T helper cell recognition of antigens shared by melanoma and glioma cells (Somasundaram R, et al: Int J Cancer 2003, 104(3):362-368).

It may be useful to measure the immune response to the peptides described herein prior to or after vaccination of a subject. A change in the immune response may be associated with or predict therapeutic benefit (Anderson M H et al: Semin Cancer Biol, 13(6):449-459. Measurement of the immune response may include quantifying amounts of cytokine induced after exposure of peripheral blood mononuclear cells in vitro to one or more of the peptides described herein. Cytokines may include those associated with Th1 and/or Th2 immune responses, including IFN-γ and IL-5, respectively.

Dogs are an animal that may benefit from treatment with the described peptides as they suffer from a high incidence of primary brain tumors (Stoica G et al: Vet Pathol, 48(1):266-275. In dogs, certain breeds have increased predispositions for malignant gliomas, including Boxers and Boston Terriers. In addition to therapeutic vaccination of dogs with a malignant glioma, prophylactic diagnostic testing with the peptides described herein may aid in earlier diagnosis and more efficacious treatment.

In some embodiments, the compositions are administered parenterally (e.g., via intramuscular injection). In some embodiments, the parenteral compositions include a vesicle that comprises a lipid. In some embodiments, the parenteral compositions include a TLR adjuvant. In some embodiments at least a portion of the TLR adjuvant present in the parenteral composition is physically associated with the vesicle.

### BRIEF DESCRIPTION OF THE DRAWINGS

The drawings described herein are for illustrative purposes only of selected embodiments and not all possible implementations, and are not intended to limit the scope of the present disclosure.

FIG. 1 illustrates Global T cell cytokine profiles among patients with CNS tumors and healthy controls.

FIG. 2 shows Memory T cell responses detected against GBM peptide antigens detected by ELISPOT.

FIG. 3 depicts the T cell cytokine profiles to each peptide among each cohort.

FIG. 4 demonstrates Th1/2 ratios of T cell responses to each peptide among each cohort.

### DEFINITIONS

Throughout the present application, several terms are employed that are defined in the following paragraphs. A non-limiting discussion of terms and phrases intended to aid understanding of the present technology is in the following paragraphs.

As used herein, the term “immune response” refers to a response elicited in an animal, including humans. An immune response may refer to cellular immunity, humoral immunity or may involve both. An immune response may also be limited to a part of the immune system. For example, in some embodiments, an immunogenic composition may induce an increased IFN-γ response. In some embodiments, an immunogenic composition may induce a systemic IgG response (e.g., as measured in serum).

As used herein, the term “immunogenic” means capable of producing an immune response in a host animal against a tumor-associated antigen (e.g., MAGE or IL-13 receptor oc2). In some embodiments, this immune response forms the basis of the therapeutic immunity elicited by a vaccine against a tumor (e.g., malignant glioma).

As used herein, the term “peptide” refers to a string of at least three amino acids linked together by peptide bonds. In general, there is no upper limit on the number of amino acids in a peptide. A peptide will generally contain only natural amino acids; however, non-natural amino acids (i.e., amino acids that do not occur in nature but that can be incorporated into a polypeptide chain) may be included. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In various embodiments, the modification(s) lead to a more stable peptide (e.g., greater half-life in vivo). Suitable modifications may include cyclization of the peptide, the incorporation of D-amino acids, etc. In various embodiments, the modification(s) lead to a more immunogenic peptide. Suitable modifications may include covalent attachment of one or more lipids (e.g., without limitation, palmitoyl, myristoyl, stearoyl, lauroyl, octanoyl, decanoyl, etc.), fusion to a carrier protein (e.g., without limitation, purified protein derivative of tuberculin (PPD), tetanus toxoid, cholera toxin and its B subunit, ovalbumin, bovine serum albumin, soybean trypsin inhibitor, muramyldipeptide and analogues thereof, a cytokine or fragment thereof, etc.), etc.

As used herein, the terms “percentage homology” refer to the percentage of sequence identity between two sequences after optimal alignment as defined in the present application. Two amino acid sequences are said to be “identical” if the sequence of amino acids in the two sequences is the same when aligned for maximum correspondence as described below. Sequence comparisons between two amino acid sequences are typically performed by comparing sequences of two optimally aligned sequences over a region or “comparison window” to identify and compare regions of sequence similarity. Optimal alignment of sequences for comparison may be conducted by the local homology algorithm of Smith and Waterman, Ad. App. Math. 2:482 (1981), by the homology alignment algorithm of Neddleman and Wunsch, J. Mol. Biol. 48:443 (1970), by the search for similarity method of Pearson and Lipman, Proc. Natl. Acad. Sci. USA 85:2444 (1988), by computerized implementation of these algorithms, or by visual inspection.

“Percentage of sequence identity” is determined by comparing two optimally aligned sequences over a comparison window, where the portion of the amino acid sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. This definition of sequence identity given above is the definition that would be used by one of ordinary skill in the art. The definition by itself does not need the help of any algorithm. The algorithms are only helpful to facilitate the optimal alignments of sequences, rather than calculate sequence identity. From this definition, it follows that there is a well defined and only one value for the sequence identity between two compared sequences which value corresponds to the value obtained for the optimal alignment.

As used herein, the terms “therapeutically effective amount” refer to the amount sufficient to show a meaningful benefit in a subject being treated. The therapeutically effective amount of an immunogenic composition may vary depending on such factors as the desired biological endpoint, the nature of the composition, the route of administration, the health, size and/or age of the subject being treated, etc.

As used herein, the term “treat” (or “treating”, “treated”, “treatment”, etc.) refers to the administration of an immunogenic composition to a subject who has a tumor, a symptom of a tumor or a predisposition toward developing a tumor, with the purpose to alleviate, relieve, alter, ameliorate, improve or affect the tumor, a symptom or symptoms of the tumor, or the predisposition towards the tumor. In some embodiments, the term “treating” refers to the vaccination of a subject.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom.

The present application provides compositions and methods useful for treating malignant gliomas. As described herein, the compositions and methods are based on the development of peptides and peptide antigens, which exhibit immunogenic properties against malignant gliomas. As described herein, the compositions and methods are based on the development of peptides and peptide combinations that detect T cells associated with cancer. In certain embodiments, these peptides have been shown to detect T cells in subjects with cancer that secrete cytokines not associated with protection against cancer.

In some embodiments, the compositions are administered parenterally (e.g., via intramuscular injection). In some embodiments, the parenteral compositions include a vesicle that comprises a lipid. In some embodiments, the parenteral compositions include a TLR adjuvant. In some embodiments at least a portion of the TLR adjuvant present in the parenteral composition is physically associated with the vesicle.

### I. Peptides

In one aspect, the present application provides peptides that can be used alone or in combination to produce an immunogenic composition for treating cancer. It is to be understood that any of these peptides may be included in an immunogenic composition and that the present application encompasses compositions that include any permutation or combination of these peptides. Section II below describes some exemplary combinations.

### Melanoma-Associated Antigen (MAGE) Peptides

Tables 1-4 describe the amino acid sequences of several peptides that have been derived from MAGE proteins.

In certain embodiments, the present application provides peptides that comprise at least 16 contiguous amino acids of SEQ ID NO. 1 (Table 1). In certain embodiments, a peptide may comprise 49 or fewer contiguous amino acids of SEQ ID NO. 1. In certain embodiments, the sequence identity may be at least 75%, 80%, 85%, 90% or 95%.

In certain embodiments, the present application provides peptides that comprise at least 16 contiguous amino acids of SEQ ID NO. 1 (see Table 2, 3). In certain embodiments, a peptide may comprise at least 15 or 16 contiguous amino acids of SEQ ID NO. 1. In certain embodiments, the sequence identity may be at least 75%, 80%, 85%, 90% or 95%.

In certain embodiments, the present application provides peptides that comprise at least 18 contiguous amino acids of SEQ ID NO. 1 (see Table 4). In certain embodiments, the sequence identity may be at least 75%, 80%, 85%, 90% or 95%.

TABLE 1
SEQ ID NO: 1

| Peptide Name | Sequence |
|---|---|
| MAGE-A3₁₁₂₋₁₆₀ | KVAELVHFLLLKYRAREPVTKAEMLGSVVGN |
| | WQYFFPVIFSKASSSLQL |

TABLE 2
SEQ ID NO: 3

| Peptide Name | Sequence |
|---|---|
| MAGE-A3₁₁₂₋₁₂₇ | KVDELAHFLLRKYRAK |

TABLE 3
SEQ ID NO: 4

| Peptide Name | Sequence |
|---|---|
| MAGE-A3₁₂₁₋₁₃₆ | LRKYRAKELVTKAEML |

TABLE 4
SEQ ID NO: 5

| Peptide Name | Sequence |
|---|---|
| MAGE-A3₁₄₃₋₁₆₀ | WQYFFPVIFSKASSSLQL |

### IL-13 Receptor Alpha 2 Peptides

Tables 5-7 describe the amino acid sequences of several peptides that have been derived from the IL-13 receptor alpha 2 (IL-13Ralpha2) protein.

In certain embodiments, the present application provides peptides that comprise at least 15 contiguous amino acids of SEQ ID NO. 2 (Table 5). In certain embodiments, a peptide may comprise 25 or fewer contiguous amino acids of SEQ ID NO. 2. In certain embodiments, the sequence identity may be at least 75%, 80%, 85%, 90% or 95%.

In certain embodiments, the present application provides peptides that comprise at least 15 contiguous amino acids of SEQ ID NO. 2 (see Table 6 and 7). In certain embodiments, the sequence identity may be at least 75%, 80%, 85%, 90% or 95%.

TABLE 5
SEQ ID NO: 2

| Peptide Name | Sequence |
|---|---|
| IL-13Ra2₃₄₁₋₃₆₅: | LLRFWLPFGFILILVIFVTQLLLRK |

TABLE 6
SEQ ID NO: 6

| Peptide Name | Sequence |
|---|---|
| IL-13Ra2₃₄₁₋₃₅₅ | LLRFWLPFGFILILV |

TABLE 7
SEQ ID NO: 7

| Peptide Name | Sequence |
|---|---|
| IL-13Ra2₃₅₁₋₃₆₅ | ILILVIFVTQLLLRK |

### II. Peptide Combinations

In one aspect, the present application provides immunogenic compositions that include combinations of peptides described in Section I. It is to be understood that the following exemplary combinations are non-limiting and that the present application encompasses all permutations and combinations of the peptides described in Section I. It is also to be understood that other peptides (present in additional MAGE proteins or other cancer testes antigens more frequently expressed in cancers) may be added to any of the immunogenic compositions described herein. In certain embodiments, each peptide in an immunogenic composition is independently immunogenic.

In certain embodiments, the present application provides immunogenic compositions that include one or more MAGE peptides from Section I.

In certain embodiments, the present application provides immunogenic compositions that include one or more IL-13Ralpha2 peptides from Section I.

In certain embodiments, the present application provides immunogenic compositions that include one or more MAGE peptides and one or more IL-13Ralpha2 peptides from Section I.

### III. Peptide Synthesis

Peptides that are described herein may be synthesized using any known method in the art (including recombinant methods). In various embodiments, peptides may be synthesized by solid phase peptide synthesis (SPPS). In SPPS, the C-terminal amino acid is attached to a solid phase (typically a cross-linked resin such as a polystyrene or polyethylene glycol-containing resin) via an acid labile bond with a linker molecule. The solid phase used is generally insoluble in the solvents used for synthesis, making it relatively simple and fast to wash away excess reagents and by-products. The N-terminus is protected with a protecting group (e.g., an Fmoc group) which is stable in acid, but removable by base. Side chain functional groups are protected with base stable, acid labile groups. The SPSS technique then involves incorporating N-α-protected amino acids into the growing peptide chain while the C-terminus remains attached to the solid phase. Example 1 describes an exemplary SPSS process.

The following references describe some exemplary methods for preparing peptide mixtures: Houghten, Proc. Natl. Acad. Sci. USA 82:5131 (1985); Geysen et al, Proc. Natl. Acad. Sci. USA 81:3998 (1984) and U.S. Pat. No. 5,010,175.

### IV. Adjuvants

In some embodiments, immunogenic compositions may include one or more adjuvants. As is well known in the art, adjuvants are agents that enhance immune responses. Adjuvants are well known in the art (e.g., see “Vaccine Design: The Subunit and Adjuvant Approach”, Pharmaceutical Biotechnology, Volume 6, Eds. Powell and Newman, Plenum Press, New York and London, 1995).

Exemplary adjuvants include complete Freund's adjuvant (CFA), incomplete Freund's adjuvant (IFA), squalene, squalane and alum (aluminum hydroxide), which are materials well known in the art, and are available commercially from several sources. In some embodiments, aluminum or calcium salts (e.g., hydroxide or phosphate salts) may be used as adjuvants. Alum (aluminum hydroxide) has been used in many existing vaccines. Typically, about 40 to about 700 μg of aluminum can be included per dose.

In various embodiments, oil-in-water emulsions or water-in-oil emulsions can also be used as adjuvants. For example, the oil phase may include squalene or squalane and a surfactant. In various embodiments, non-ionic surfactants such as the mono- and di-C₁₂-C₂₄-fatty acid esters of sorbitan and mannide may be used. The oil phase preferably comprises about 0.2 to about 15% by weight of the immunogenic peptide(s) (e.g., about 0.2 to 1%). PCT Publication No. WO 95/17210 describes exemplary emulsions.

The adjuvant designated QS21 is an immunologically active saponin fractions having adjuvant activity derived from the bark of the South American tree Quillaja Saponaria Molina, and the method of its production is disclosed in U.S. Pat. No. 5,057,540. Semi-synthetic and synthetic derivatives of Quillaja Saponaria Molina saponins are also useful, such as those described in U.S. Pat. Nos. 5,977,081 and 6,080,725.

TLRs are a family of proteins homologous to the Drosophila Toll receptor, which recognize molecular patterns associated with pathogens and thus aid the body in distinguishing between self and non-self molecules. Substances common in viral pathogens are recognized by TLRs as pathogen-associated molecular patterns. For example, TLR-3 recognizes patterns in double-stranded RNA, TLR-4 recognizes patterns in lipopolysaccharides, TLR-7/8 recognize patterns containing adenosine in viral and bacterial RNA and DNA while TLR-9 recognizes unmethylated bacterial CpG DNA. When a TLR is triggered by such pattern recognition, a series of signaling events occurs that leads to inflammation and activation of innate and adaptive immune responses. A number of synthetic ligands containing the molecular patterns recognized by various TLRs are being developed as adjuvants and may be included in an immunogenic composition as described herein.

For example, polyriboinosinic:polyribocytidylic acid or poly(I:C) (available from InvivoGen of San Diego, Calif.) is a synthetic analog of double-stranded RNA (a molecular pattern associated with viral infection) and an exemplary adjuvant that is an agonist for TLR-3 (e.g., see Field et al., Proc. Natl. Acad. Sci. USA 58:1004 (1967) and Levy et al., Proc. Natl. Acad. Sci. USA 62:357 (1969)). In some embodiments, poly(I:C) may be combined with other agents to improve stability (e.g., by reducing degradation via the activity of RNAses). For example, U.S. Pat. Nos. 3,952,097, 4,024,241 and 4,349,538 describe poly(I:C) complexes with poly-L-lysine. The addition of poly-arginine to poly(I:C) has also been shown to reduce degradation via the activity of RNAses. U.S. Patent Publication No. 20090041809 describes double-stranded nucleic acids with one or more than one locked nucleic acid (LNA) nucleosides that can act as TLR-3 agonists. Those skilled in the art will be able to identify other suitable TLR-3 agonist adjuvants.

Attenuated lipid A derivatives (ALD) such as monophosphoryl lipid A (MPL) and 3-deacyl monophosphoryl lipid A (3D-MPL) are exemplary adjuvants that are agonists for TLR-4. ALDs are lipid A-like molecules that have been altered or constructed so that the molecule displays lesser or different of the adverse effects of lipid A. These adverse effects include pyrogenicity, local Shwarzman reactivity and toxicity as evaluated in the chick embryo 50% lethal dose assay (CELD₅₀). MPL and 3D-MPL are described in U.S. Pat. Nos. 4,436,727 and 4,912,094, respectively. MPL was originally derived from lipid A, a component of enterobacterial lipopolysaccharides (LPS), a potent but highly toxic immune system modulator. 3D-MPL differs from MPL in that the acyl residue that is ester linked to the reducing-end glucosamine at position 3 has been selectively removed. It will be appreciated that MPL and 3D-MPL may include a mixture of a number of fatty acid substitution patterns, i.e., heptaacyl, hexaacyl, pentaacyl, etc., with varying fatty acid chain lengths. Thus, various forms of MPL and 3D-MPL, including mixtures thereof, are encompassed by the present disclosure.

In some embodiments these ALDs may be combined with trehalosedimycolate (TDM) and cell wall skeleton (CWS), e.g., in a 2% squalene/Tween™ 80 emulsion (e.g., see GB Patent No. 2122204). MPL is available from Avanti Polar Lipids, Inc. of Alabaster, Ala. as PHAD (phosphorylated hexaacyl disaccharide). Those skilled in the art will be able to identify other suitable TLR-4 agonist adjuvants. For example, other lipopolysaccharides have been described in WO 98/01139; U.S. Pat. No. 6,005,099 and EP Patent No. 729473.

Imiquimod (1-isobutyl-1H-imidazo[4,5-c]quinolin-4-amine) is a small molecule agonist of TLR-7/8 which may also be advantageously included in an immunogenic composition as described herein.

Activation of Toll-like receptor 9 (TLR9) by DNA containing unmethylated CpG motifs, its natural ligand, produces potent Th1-type innate and adaptive immune responses (Hemmi, H et al: Nature 2008 408:740-745). TLR9-stimulated B cells and plasmacytoid dendritic cells secrete a number of Th-1-promoting cytokines and chemokines, including IL-12, IL-6, IFN-γ, Type 1 IFNs, MIP-1, and IP-10 (Tokunaga T et al: Microbiol Immunol 1992 36:55-66; Krieg A M: Nat Rev Drug Discov 2006 5:471-484; Kandimalla E R et al: Proc Natl Acad Sci USA 2005 102:6925-6930). Agonists of TLR9 have shown antitumor activity, alone and in combination with chemotherapy and radiotherapy, and ability to enhance the antibody-dependent cell-mediated cytotoxicity (ADCC) of mAbs in a number of preclinical and early clinical trials (Krieg A M: Nat Rev Drug Discov 2006 5:471-484; Van Ojik H H et al: Cancer Res 2003 63:5595-5600).

Based on extensive structure-activity relationship studies, synthetic agonists of TLR9 containing novel DNA structures and synthetic dinucleotide motifs, referred to as immune modulatory oligonucleotides (IMOs), have been synthesized, demonstrating distinct cytokine profiles in vitro and in vivo, compared with conventional TLR9 agonists (Kandimalla E R et al: Proc Nall Acad Sci USA 2005 102:6925-6930; Kandimalla E R et al: Proc Nall Acad Sci USA 2003 100:14303-14308; Kandimalla E R et al: Nucleic Acids Res 2003 31:2393-2400) and higher metabolic stability due to the novel DNA structure present in them (Yu D et al: Nucleic Acids Res 2002 30:4460-4469; Kandimalla E T et al: Bioconjug Chem 2002 13:966-974; Wang D et al: Vaccine 2005 23:2614-2622). Previous studies have demonstrated potent antitumor activity of IMOs as monotherapies and in combination with chemotherapeutic agents and mAbs (Wang D et al: Int J Oncol 2004 74:901-908; Damiano V et al: Clin Cancer Res 2006 12:577-583). Currently, a synthetic agonist of TLR9, IMO-2055, is under clinical evaluation, in combination with chemotherapy and other agents in cancer patients.

### V. Vesicles

In certain embodiments, one or more peptides in a composition may be associated with a vesicle. As is well known in the art, vesicles generally have an aqueous compartment enclosed by one or more bilayers which include amphipathic molecules (e.g., fatty acids, lipids, steroids, etc.). Generally, the one or more peptides will be present in the aqueous core of the vesicle. However, depending on its hydrophobicity, a peptide may also be associated with a bilayer (e.g., through hydrophobic interactions and/or hydrogen or ionic bonds). It is to be understood that any vesicle may be used with an immunogenic composition as described herein and that the amphipathic molecules of the bilayer may be ionic or non-ionic. Phospholipids are exemplary ionic molecules.

In certain embodiments, one or more peptides are associated with a vesicle that comprises a non-ionic surfactant. Any non-ionic surfactant with appropriate amphipathic properties may be used to form such a vesicle. Without limitation, examples of suitable surfactants include ester-linked surfactants based on glycerol. Such glycerol esters may comprise one of two higher aliphatic acyl groups, e.g., containing at least ten carbon atoms in each acyl moiety. Surfactants based on such glycerol esters may comprise more than one glycerol unit, e.g., up to 5 glycerol units. Glycerol monoesters may be used, e.g., those containing a C₁₂-C₂₀alkanoyl or alkenoyl moiety, for example caproyl, lauroyl, myristoyl, palmitoyl, oleyl or stearoyl. An exemplary surfactant is 1-monopalmitoyl glycerol.

Ether-linked surfactants may also be used as the non-ionic surfactant. For example, ether-linked surfactants based on glycerol or a glycol having a lower aliphatic glycol of up to 4 carbon atoms, such as ethylene glycol, are suitable. Surfactants based on such glycols may comprise more than one glycol unit, e.g., up to 5 glycol units (e.g., diglycolcetyl ether and/or polyoxyethylene-3-lauryl ether). Glycol or glycerol monoethers may be used, including those containing a C₁₂-C₂₀alkanyl or alkenyl moiety, for example capryl, lauryl, myristyl, cetyl, oleyl or stearyl. Ethylene oxide condensation products that can be used include those disclosed in PCT Publication No. WO88/06882 (e.g., polyoxyethylene higher aliphatic ether and amine surfactants). Exemplary ether-linked surfactants include 1-monocetyl glycerol ether and diglycolcetyl ether.

It is also to be understood that vesicles may also incorporate an ionic amphiphile, e.g., to cause the vesicles to take on a negative charge. For example, this may help to stabilize the vesicles and provide effective dispersion. Without limitation, acidic materials such as higher alkanoic and alkenoic acids (e.g., palmitic acid, oleic acid) or other compounds containing acidic groups including phosphates such as dialkyl phosphates (e.g., dicetylphospate, or phosphatidic acid or phosphatidyl serine) and sulphate monoesters such as higher alkyl sulphates (e.g., cetylsulphate), may all be used for this purpose.

To form vesicles, the components are generally admixed with an appropriate hydrophobic material of higher molecular mass capable of forming a bi-layer (such as a steroid, e.g., a sterol such as cholesterol). The presence of the steroid assists in forming the bi-layer on which the physical properties of the vesicle depend.

It will be appreciated that there are known techniques for preparing vesicles comprising non-ionic surfactants, such as those referred to in PCT Publication No. WO1993/019781. An exemplary technique is the rotary film evaporation method, in which a film of non-ionic surfactant is prepared by rotary evaporation from an organic solvent, e.g., a hydrocarbon or chlorinated hydrocarbon solvent such as chloroform, e.g., see Russell and Alexander, J. Immunol. 140:1274 (1988). The resulting thin film is then rehydrated in bicarbonate buffer in the presence of the transport enhancer.

Another method for the production of vehicles is that disclosed by Collins et al., J. Pharm. Pharmacol. 42:53 (1990). This method involves melting a mixture of the non-ionic surfactant, steroid (if used) and ionic amphiphile (if used) and hydrating with vigorous mixing in the presence of aqueous buffer. The transport enhancer can be incorporated into the vesicles, either by being included with the other constituents in the melted mixture or concomitantly during the process used to entrap the peptide(s).

Another method involves hydration in the presence of shearing forces. An apparatus that can be used to apply such shearing forces is a well known, suitable equipment (see, e.g., PCT Publication No. WO88/06882). Sonication and ultra-sonication are also effective means to form the vesicles or to alter their particle size.

The one or more peptides may be associated with vesicles in any manner. For example, in the rotary film evaporation technique, this can be achieved by hydration of the film in the presence of peptide(s) together with the transport enhancer. In other methods, the one or more peptides may be associated with preformed vesicles by a dehydration-rehydration method in which antigen present in the aqueous phase is entrapped by flash freezing followed by lyophilisation, e.g., see Kirby and Gregoriadis, Biotechnology 2:979 (1984). Alternatively a freeze thaw technique may be used in which vesicles are mixed with the peptide(s) and repeatedly flash frozen in liquid nitrogen, and warmed to a temperature of the order of, e.g., 60° C. (i.e., above the transition temperature of the relevant surfactant), e.g., see Pick, Arch. Biochem. Biophys. 212:195 (1981). In addition to entrapping peptides, the dehydration-rehydration method and freeze-thaw technique are also capable of concomitantly incorporating additional transport enhancers into the vesicles.

In each of these methods, the suspension of vesicle components may be extruded several times through microporous polycarbonate membranes at an elevated temperature sufficient to maintain the vesicle-forming mixture in a molten condition. This has the advantage that vesicles having a uniform size may be produced. Vesicles that may be used in accordance with the invention may be of any diameter. In certain embodiments, the composition may include vesicleswith diameter in range of about 10 nm to about 10 μm. In certain embodiments, vesicles are of diameters between about 100 nm to about 5 μm. In certain embodiments, vesicles are of diameters between about 500 nm to about 2 μm. In certain embodiments, vesicles are of diameters between about 800 nm to about 1.5 μm.

The steroid, if present, will typically comprise between 20 and 120% by weight of the non-ionic surfactant. The ionic amphiphile, if present, will typically comprise, between 1 and 30% by weight of the non-ionic surfactant.

### VI. Global T Cell Responses

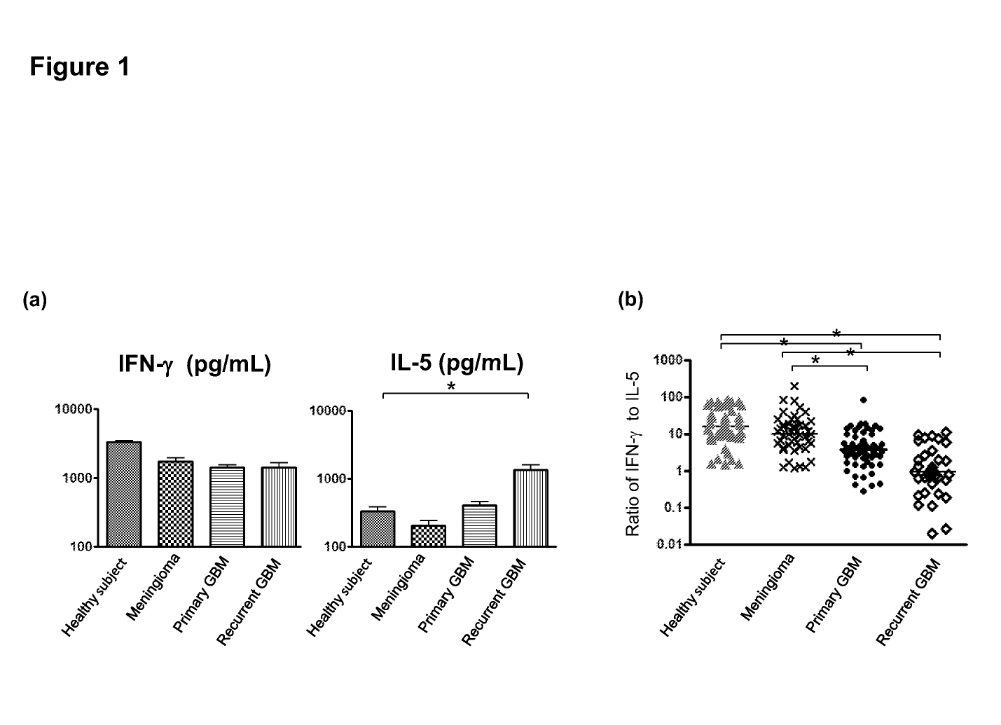

Six primary T cell cultures were established from each patient against all stimuli. Anti-CD3 mAb was used to stimulate and expand T cells to confirm T cell viability and to examine global, nonspecific T cell cytokine responses among the different cohorts. Relative to healthy subjects, anti-CD3 mAb-induced IFN-γ levels in patients with GBMs (primary and recurrent) and meningiomas were modestly lower (FIG. 1a). More strikingly, anti-CD3 mAb stimulation uniquely induced secretion of high amounts of IL-5 from patients with recurrent GBMs (P<0.0001). A recent clinical trial examined the IFN-γ/IL-5 ratio after polyclonal stimulation of PBMCs in patients with metastatic melanoma treated with immunomodulators given to restore the Th1/Th2 balance (Green D S, et al: Br J Dermatol 2008, 159(3):606-614). A similar analysis is provided of the data presented herein (FIG. 1b). The IFN-γ/IL-5 ratios in both primary GBM patients (geometric mean 3.7) and recurrent GBMs (geometric mean 0.9) were significantly lower than those in healthy subjects (geometric mean 16.0) and meningioma patients (geometric mean 10.0) (p<0.001). This antigen-nonspecific bias towards a Th2 response in patients with primary and recurrent GBMs is consistent with past reports (Driessens G, et al: Cancer Immunol Immunother 2008, 57(12):1745-1756; Kumar R, et al: Oncol Rep 2006, 15(6):1513-1516; Li G, et al: Chin Med Sci J2005, 20(4):268-272; Roussel E, et al: Clin Exp Immunol 1996, 105(2):344-352). There was no significant difference in the global IFN-γ/IL-5 ratio between healthy subjects and meningioma patients, indicating that neither treatment with steroids or antiepileptic medications nor the simple presence of a CNS tumor were responsible for the deviation in global T cell responses.

### VII. Responses to HLA Class II-Restricted Peptide Stimulation

Both glial cells and melanocytes derive from neural ectoderm (Lallier T E: Ann N Y Acad Sci 1991, 615:158-171) and several studies have demonstrated that melanoma-associated tumor antigens are also expressed by gliomas, including MAGE-A3 (Chi D D, et al: Am J Pathol 1997, 150(6):2143-2152; Sahin U, et al: Clin Cancer Res 2000, 6(10):3916-3922; Saikali S, et al: J Neurooncol 2007, 81(2):139-148). Like MAGE-A3, IL-13Rα2 is a cancer testes antigen that is over-expressed in gliomas (Debinski W, et al: Clin Cancer Res 1999, 5(5):985-990; Mintz A, Debinski W: Crit Rev Oncog 2000, 11(1):77-95). In trying to identify novel glioma-associated HLA class II-restricted T helper cell epitopes, epitopes identified in patients with melanoma were assessed for similar expression by patients with gliomas. Two such epitopes with homology to MAGE-A3 were identified (Chaux P, et al: J Exp Med 1999, 189(5):767-778; Kobayashi H, et al: Cancer Res 2001, 61(12):4773-4778; Manici S, et al: J Exp Med 1999, 189(5):871-876), and modified to incorporate adjacent HLA class I-restricted CTL epitopes (Kawashima I, et al: Hum Immunol 1998, 59(1):1-14; Miyagawa N, et al: Oncology 2006, 70(1):54-62; Russo V, et al: Proc Natl Acad Sci USA 2000, 97(5):2185-2190; Schultz E S, et al: J Exp Med 2002, 195(4):391-399) as well as several amino acid substitutions. The amino acid substitutions altered the hydrophobicity of the peptides but not their charge (Ala to Asp, Leu to Arg) and potentially their secondary structure (Pro to Leu). Similarly, two overlapping 15mer IL-13Rα2 epitopes were identified, one of which was modified to incorporate a CTL epitope (Okano F, et al: Clin Cancer Res 2002, 8(9):2851-2855). The five epitopes used in this study in relation to previously described epitopes are depicted in Tables 1-7.

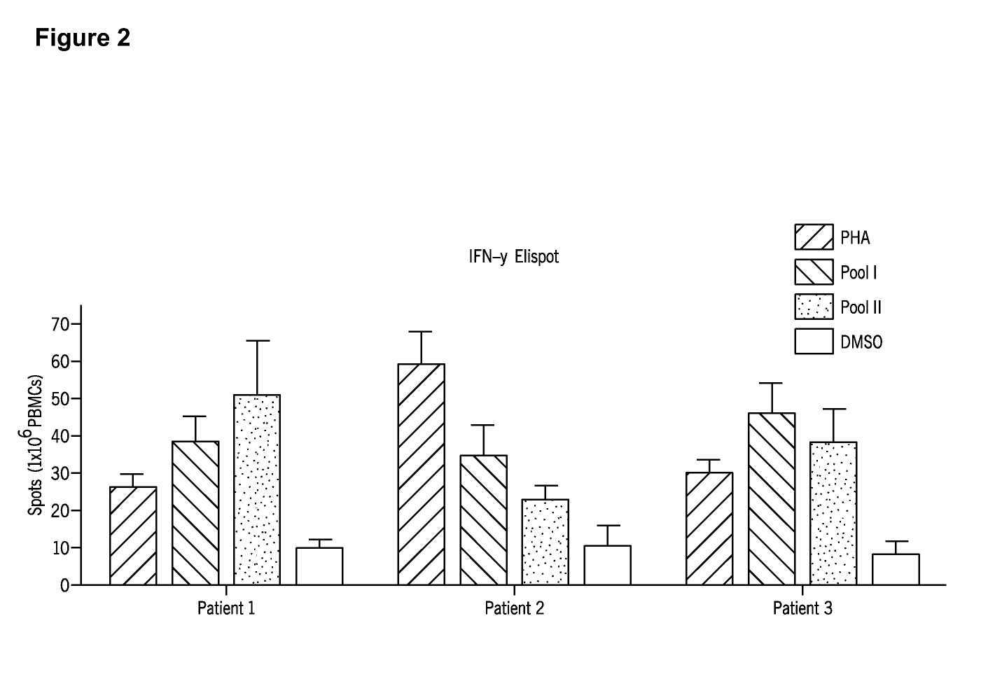

Measurement of antigen-specific T cell responses in the peripheral blood in humans differs depending on whether responses are high affinity interactions with foreign (viral) epitopes or lower affinity interactions involving recognition of self-antigens. It has been previously demonstrated that high frequencies of T cells directed against the self-antigen MBP peptide 85-99 in the peripheral blood of patients with multiple sclerosis (MS) fail to proliferate when stimulated with antigen but readily secrete high levels of cytokine (Windhagen A, et al: J Neuroimmunol 1998, 91(1-2):1-9). Given that T cell responses directed against MAGE and IL-13Rα2 antigens also involve T cells with low affinity to these self-antigens, antigen-specific responses were quantified based on cytokine secretion, as recently described in a phase I study of patients with MS (Viglietta V, et al: Neurology 2008, 71(12):917-924). Cytokine production was quantified by ELISA, defining a positive T cell response for each patient as the amounts of IFN-γ or IL-5 that were >50 pg/mL and two standard deviations above the mean cytokine levels secreted after stimulation of cells from that patient with negative control MBP peptide. The mean cut-off for a positive cytokine response based on cytokine induced by stimulation with control MBP peptide was 895 pg/ml (range: 13-1298) and 314 pg/ml (range: 72-852) for IFN-γ and IL-5 among healthy subjects, and was 123 pg/ml (range: 0-286) and 312 pg/ml (range: 59-1347) for IFN-γ and IL-5 among GBM patients. Use of a traditional IFN-γ ELISPOT assay, in which quantification of spots can at times be ambiguous, confirmed that memory T cell responses could be detected with these peptides in patients with primary GBMs, as peptide specific cytokine production could be detected within 48 hours of culture (FIG. 2).

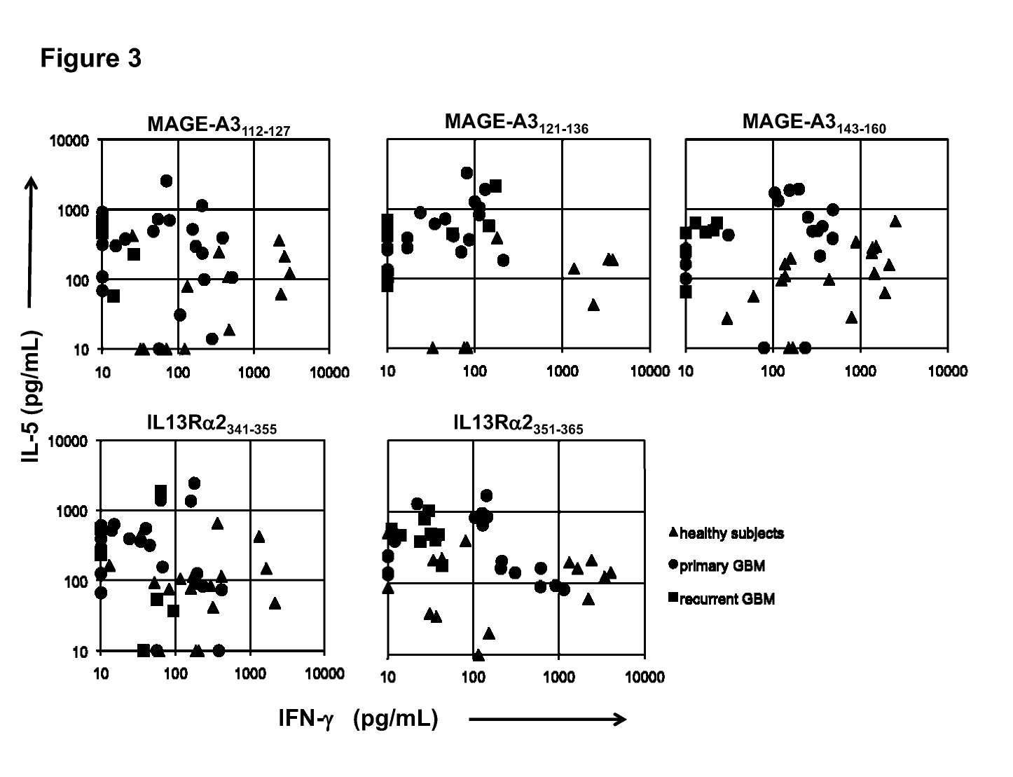

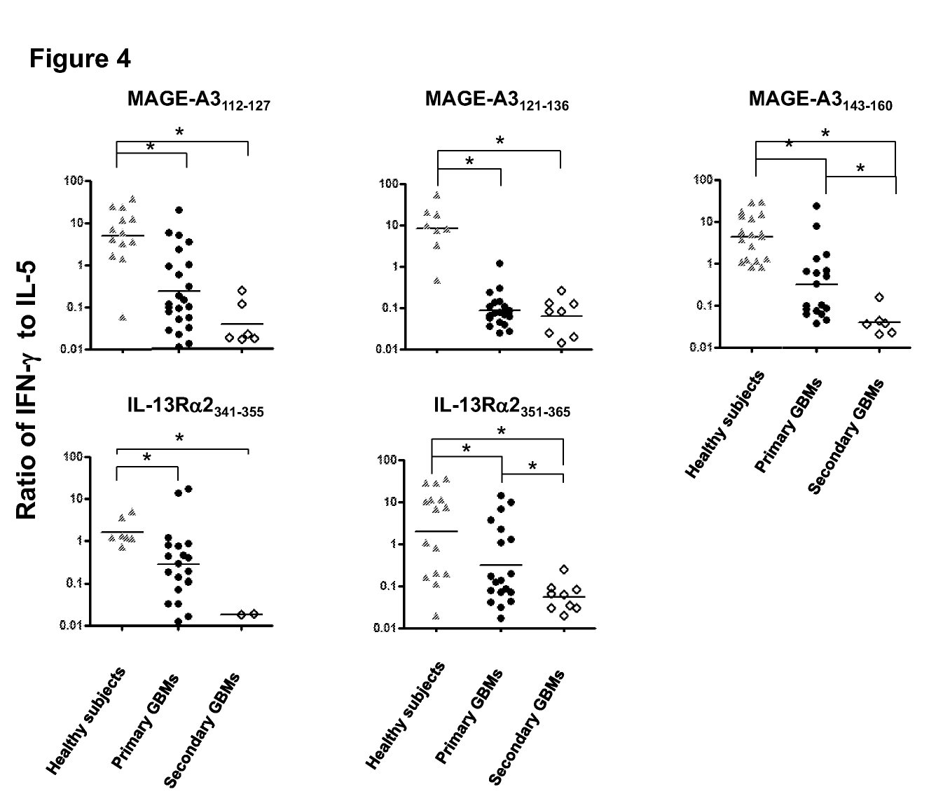

T cells responding to all five peptides examined among healthy subjects exhibited a predominant Th1 response (high IFN-γ and low IL-5 secretion) (FIG. 3). In marked contrast, the majority of peptide-specific T cell responses among both primary and recurrent GBM patients were Th2 polarized (low IFN-γ and high IL-5 secretion). Frequencies of response to the individual peptides were most prevalent among healthy subjects and patients with primary GBMs, both in terms of the number of subjects responding to a given peptide and the number of positive lines. Responses in one or a few subjects did not dominate among any of the cohorts examined; at least half of the subjects (in some cases all) in each cohort responded to all of the epitopes tested (Table 10). Patients with meningiomas generally had less frequent responses, though strong responses could be detected against both MAGE-A3 and IL-13Rα2 peptides. Mean IFN-γ/IL-5 ratios were significantly lower (p<0.05) for patients with primary GMBs (geometric means for MAGE-A3₁₁₂₋₁₂₇, MAGE-A3₁₂₁₋₁₃₆, MAGE-A3₁₄₃₋₁₆₀, IL13Rα2₃₄₁₋₃₅₅, and IL-13Rα2₃₅₁₋₃₆₅ were 0.2, 0.1, 0.3, 0.3, 0.3, respectively) relative to healthy subjects (geometric means were 4.9, 8.1, 4.3, 1.6, 2.0, respectively) in response to all of the epitopes (FIG. 4). The Th2 bias was even more profound among patients with recurrent GBMs (geometric means for MAGE-A3₁₁₂₋₁₂₇, MAGE-A3₁₂₁₋₁₃₆, MAGE-A3₁₄₃₋₁₆₀, IL13Rα2₃₄₁₋₃₅₅, and IL-13Rα2₃₅₁₋₃₆₅ were 0.04, 0.06, 0.4, 0.02, 0.05), and was significantly lower than that of patients with primary GBMs for the MAGE-A3₁₄₃₋₁₆₀ and IL-13Rα2₃₅₁₋₃₆₅ epitopes (P<0.05).

### VIII. Dosage and Administration

The methods described herein are useful for treating malignant gliomas in humans including adults and children. In general however they may be used with any animal. In some embodiments, the methods herein may be used for veterinary applications, e.g., canine applications.

Compositions described herein will generally be administered in such amounts and for such a time as is necessary or sufficient to induce an immune response. Dosing regimens may consist of a single dose or a plurality of doses over a period of time. The exact amount of a peptide composition to be administered may vary from subject to subject and may depend on several factors. Thus, it will be appreciated that, in general, the precise dose used will be as determined by the prescribing physician and will depend not only on the weight of the subject and the route of administration, but also on the age of the subject and the severity of the symptoms and/or the risk of infection. In some embodiments, the dose of peptide in an immunogenic composition may range from about 0.01 to 50 mg. For example, in some embodiments the range may be between 0.1 and 5 mg, e.g., between 0.1 and 2 mg.

In some embodiments, the compositions may be formulated for delivery parenterally, e.g., by injection. In such embodiments, administration may be, for example, intravenous, intramuscular, intradermal, or subcutaneous, or via by infusion or needleless injection techniques. For such parenteral administration, the compositions may be prepared and maintained in conventional lyophylized compositions and reconstituted prior to administration with a pharmaceutically acceptable saline solution, such as a 0.9% saline solution. The pH of the injectable composition can be adjusted, as is known in the art, with a pharmaceutically acceptable acid, such as methanesulfonic acid. Other acceptable vehicles and solvents that may be employed include Ringer's solution and U.S.P. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable compositions can be sterilized, for example, by filtration through a bacterial-retaining filter, or by incorporating sterilizing agents in the form of sterile solid compositions which can be dissolved or dispersed in sterile water or other sterile injectable medium prior to use.

In some embodiments, parenteral compositions comprise one or more peptides and an adjuvant. In some embodiments, the adjuvant is a TLR agonist.

### IX. Exemplary Compositions

In one aspect, the present application provides immunogenic compositions that include combinations of peptides described in example 1. It is to be understood that the following exemplary combinations are non-limiting and that the present application encompasses all permutations and combinations of the peptides described in example 1. In certain embodiments, each peptide in an immunogenic composition is independently immunogenic. In some embodiments, the immunogenic compositions comprise one or more peptides comprising a region having at least 75%, 80%, 85%, 90% or 95% sequence identity with 16-49 contiguous amino acids of SEQ ID NO. 1, wherein the one or more peptides comprise 49 or fewer contiguous amino acids from MAGE A3 protein. In some embodiments, the immunogenic compositions comprise one or more peptides comprising a region having at least 75%, 80%, 85%, 90% or 95% sequence identity with 16-25 contiguous amino acids of SEQ ID NO. 2, wherein the one or more peptides comprise 25 or fewer contiguous amino acids from IL-13Rα2 protein. In some embodiments, the immunogenic compositions comprise one or more peptides comprising a region having at least 75%, 80%, 85%, 90% or 95% sequence identity with 16-49 contiguous amino acids of SEQ ID NO. 1, wherein the one or more peptides comprise 49 or fewer contiguous amino acids from MAGE A3 protein and one or more peptides comprising a region having at least 75%, 80%, 85%, 90% or 95% sequence identity with 16-25 contiguous amino acids of SEQ ID NO. 2, wherein the one or more peptides comprise 25 or fewer contiguous amino acids from IL-13Rα2 protein.

In some embodiments, the present disclosure provides immunogenic compositions that include a TLR-4 agonist adjuvant. In some embodiments, these compositions may be administered parenterally (e.g., by intramuscular injection). In some embodiments the TLR-4 agonist adjuvant comprises monophosphoryl lipid A or 3-deacyl monophosphoryl lipid A. In some embodiments, the composition further comprises alum or lipids that form vesicles.

In some embodiments, the present disclosure provides immunogenic compositions that include a TLR-9 agonist adjuvant. In some embodiments, these compositions may be administered parenterally (e.g., by intramuscular injection). In some embodiments the TLR-9 agonist adjuvant comprises Immune Modulatory Oligonucleotides (IMO 2055). In some embodiments, the composition further comprises alum or lipids that form vesicles.

In some embodiments, the aforementioned compositions are used to diagnose an individual or animal suffering from, or at risk for, Glioblastoma.

In some embodiments, the aforementioned compositions are used to treat an individual or animal suffering from, or at risk for, Glioblastoma.

### EXAMPLES

The following examples describe some exemplary modes of making and practicing certain compositions and methods that are described herein. It should be understood that these examples are for illustrative purposes only and are not meant to limit the scope of the compositions and methods described herein.

### Example 1

### Peptides and Peptide Synthesis

All peptides were synthesized using standard FMOC chemistry to 95% purity (New England Peptide Company). All peptides were at least 15 amino acids in length. The following peptides were used for stimulation of PBMCs: MAGE-A3₁₁₂₋₁₂₇ (KVDELAHFLLRKYRAK) (SEQ ID NO: 3); MAGE-A3₁₂₁₋₁₃₆ (LRKYRAKELVTKAEML) (SEQ ID NO: 4); MAGE-A3₁₄₃₋₁₆₀ (WQYFFPVIFSKASSSLQL) (SEQ ID NO: 5); IL13Rα2₃₄₁₋₃₅₅ (LLRFWLPFGFILILV) (SEQ ID NO: 6); IL-13Rα2₃₅₁₋₃₆₅ (ILILVIFVTGLLLRK) (SEQ ID NO: 8); MBP₈₅₋₉₉ (ENPVVHFFKNIVTPR) (SEQ ID NO: 9). HLA class II alleles predicted to bind the peptides were determined using ProPred HLA class II binding algorithm (Singh H, Raghava G P, Bioinformatics 2001, 17(12):1236-1237), summarized in Table 8. All peptides were predicted to be very promiscuous, binding to multiple (up to 9 in several cases) alleles. The validity of the predictions is supported by experimental data for the MBP₈₅₋₉₉ peptide, which has been shown to bind to both the DRB1*04 and *15 alleles as predicted (Mycko M P, Waldner H, Anderson D E, Bourcier K D, Wucherpfennig K W, Kuchroo V K, Hafler D A, J Immunol 2004, 173(3):1689-1698).

TABLE 8

| Epitope | Predicted HLA Class II Binding Alleles |
|---|---|
| MAGE-A3₁₁₂₋₁₂₇ | DRB1*04, *08, *11, *13, DRB5*01 |
| MAGE-A3₁₂₁₋₁₃₆ | DRB1*04, *07, *08, *11, *13, *15, DRB5*01 |
| MAGE-A3₁₄₃₋₁₆₀ | DRB1*01, *03, *04, *07, *08, *11, *13, *15, |
| | DRB5*01 |
| IL-13Ra2₃₄₁₋₃₅₅ | DRB1*01, *03, *04, *07, *08, *11, *13, *15, |
| | DRB5*01 |
| IL-13Ra2₃₅₁₋₃₆₅ | DRB1*01, *03, *04, *07, *08, *11, *13, *15, |
| | DRB5*01 |
| MBP₈₅₋₉₉ | DRB1*01, *03, *04, *08, *11, *13, *15 |

Candidate glioma-associated T helper epitopes are depicted in Table 9. The location of five candidate glioma-associated epitopes are depicted within the MAGE-A3 and IL-13Rα2 protein sequences. The location of documented melanoma-associated CTL epitopes are highlighted by underlining within the three candidate MAGE-A3 epitopes and one of the IL-13Rα2 peptides. The location of previously described HLA class II-restricted melanoma epitopes (MAGE-A3₁₂₁₋₁₃₄ and MAGE-A3₁₄₆₋₁₆₀) are shown for comparative purposes. Amino acid differences in the candidate glioma-associated epitopes from the MAGE-A3 sequence are highlighted in bold.

TABLE 9
MAGE-A3₁₁₂₋₁₆₀:
(SEQ ID NO: 1)
KVAELVHFLLLKYRAREPVTKAENALGSVVGNWQYFFPVIFSRASSSLQL
MAGE-A3₁₁₂₋₁₂₇:
(SEQ ID NO: 3)
KVDELAHFLLRKYRAK
MAGE-A3₁₂₁₋₁₃₆:
(SEQ ID NO: 4)
LRKYRAKELVTKAEML
MAGE-A3₁₄₃₋₁₆₀:
(SEQ ID NO: 5)
WQYFFPVIFSKASSSLQL
MAGE-A3₁₂₁₋₁₃₄:
(SEQ ID NO: 10)
FLLLKYRAREPVTKAE
MAGE-A3₁₄₆₋₁₆₃:
(SEQ ID NO: 11)
FFPVIFSKASSSLQL
IL-13Ra2₃₄₁₋₃₆₅:
(SEQ ID NO: 2)
LLRFWLPFGFILILVIFVTQLLLRK
IL-13Ra2₃₄₁₋₃₅₅:
(SEQ ID NO: 6)
LLRFWLPFGFILILV
IL-13Ra2₃₅₁₋₃₆₅:
(SEQ ID NO: 7)
ILILVIFVTQLLLRK

### Example 2

### Isolation of PBMCs, Culture with Peptides and Cytokine Measurement

Twenty-five mL of blood from patients or healthy subjects was obtained under an IRB-approved protocol. All samples of blood from patients were taken at the time of surgery. Ages of the patients ranged from 48 to 76 among patients with primary GBMs (median age 55), from 41 to 69 among patients with recurrent GBMs (median age 52), and from 40 to 73 among patients with meningiomas (median age 62). A greater number of men had primary or secondary GBMs (6 of 8 patients and 5 of 6 patients, respectively), while more women than men had meningiomas (5 of 8 patients). Primary and recurrent GBMs were located in temporal, parietal, and frontal lobes with comparable frequencies. All tumor-bearing patients received similar doses of steroids and anti-epileptic medications at the time of tumor debulking surgery prior to obtaining peripheral blood for these studies. T cell responses in patients with meningiomas controlled for influences of steroids on antigen responsiveness and cytokine balance. Tumor tissue was independently confirmed in all cases by formal pathological analysis. PBMCs were purified from heparinized blood by density gradient centrifugation using Ficoll-Hypaque (GE Healthcare Biosciences), and cells were then washed with PBS and viable cells quantified by trypan-blue staining.

Freshly isolated PBMCs were plated at 2×10⁵ cells/well in 200 μl of serum-free X-VIVO15 (X15) media (Lonza) in 96-well round-bottom cell culture plates. Candidate peptides, in addition to a negative control peptide derived from MBP were added at a concentration of 10 μg/mL and anti-CD3 mAb was added at a concentration of 1 μg/mL. Six T cell cultures were established for each condition in each subject, and 100 IU/ml of IL-2 was added on the following day. Plates were incubated at 37° C. and 5% CO₂ for 14 days, with media changed as needed, and the supernatant was harvested to evaluate T cell responses (cytokines) induced by each condition using ELISA. In a limited number (n=3) of patients, cytokine production was assessed after both 7 and 14 days. Tumor-specific responses were apparent at day 7, and the frequency of positive responses did not change significantly at day 14, but the cytokine values did increase significantly (data not shown). An IFN-γ ELISPOT assay was performed as previously described (Lv H, Havari E, et al: J Clin Invest, 121(4):1561-1573).

To detect T helper cell responses directed against the candidate peptides, IFN-γ and IL-5 were measured by ELISA using commercially available kits supplied by BD bioscience. IFN-γ was used as a prototypic Th1 cytokine and IL-5 was chosen as a prototypic cytokine released by Th2 cells because unlike IL-4 there would be no potential consumption by antigen-specific T cells in the culture conditions (Kourilsky P, Truffa-Bachi P: Trends Immunol 2001, 22(9):502-509). The Th2-associated transcription factor GATA-3 directly binds and regulates both the IL-4 and IL-5 gene promoters (Zhou M, Ouyang W: Immunol Res 2003, 28(1):25-37) and a positive correlation has been reported among GATA-3, IL-4, and IL-5 gene expression during human T cell differentiation (Lantelme E, et al: Immunology 2001, 102(2):123-130), providing further support for analysis of IL-5 as a representative Th2 cytokine. Initial experiments also examined the secretion of IL-10 in response to peptide stimulation, which was not detected. Flat-bottom microtiter plates (Immulon) were coated with primary antibody (IFN-γ or IL-5) diluted 1:1000 in NaHCO3 and incubated overnight at 4° C. Coating solution was then removed, plates blocked with PBS+10% FBS at 25° C. for 2 hours, rinsed 3 times with diluted wash buffer (dH2O, Tween 20, PBS 20×), and standards were then added in duplicate at 0, 62.5, 125, 250, 500, 1000, 2000, and 4000 pg/mL (diluted in X15 media). Supernatants (50 μl/well) from T cell assays were then added to wells. Plates were incubated for 2 hours at 25° C. and subsequently rinsed 3 times. Wells were then coated with a secondary biotinylated antibody diluted 1:1000 in PBS+1% FBS and incubated for 1 hour at 25° C. Plates were again rinsed 3 times and avidin-peroxidase diluted 1:1000 in PBS+10% FBS was added and incubated for another 1 hour. After rinsing 6 times, TMB (tetramethylbenzidine) (BD biosciences) was added to wells, which were allowed to develop. The reaction was stopped by adding 50 μL of sulfuric acid and absorbance was measured at 455 nm by an ELISA plate reader (BIO-RADR). A standard curve was generated by plotting absorbance against each reference standard, and sample concentrations were extrapolated from this curve. Appropriate statistical tests and analyses based on the data were determined using Prism 5.0 (GraphPad software).

### Example 3

### Global T Cell Cytokine Profiles Among Patients with CNS Tumors and Healthy Controls

FIG. 1 illustrates Global T cell cytokine profiles among patients with CNS tumors and healthy controls. (a) The geometric mean values and standard deviation of IFN-γ and IL-5 levels from all T cell cultures generated with anti-CD3 mAb among the four groups examined are presented. Modest decreases in the amount of secreted IFN-γ are seen among all patients with CNS tumors when compared to healthy subjects, while a significant elevation of IL-5 levels is seen only in recurrent GBM patients. (b) The ratios of IFN-γ to IL-5 for all primary T cell responses are shown for each cohort. There was no difference in this ratio comparing patients with meningiomas to healthy subjects, but patients with primary and recurrent GBM patients exhibited significantly lower ratios compared to both healthy subjects and meningioma patients.

### Example 4

### Memory T Cell Responses Detected Against GBM Peptide Antigens Detected by ELISPOT

FIG. 2 shows Memory T cell responses detected against GBM peptide antigens detected by ELISPOT. The MAGE-AE peptides (MAGE-A3₁₁₂₋₁₂₇, MAGE-A3₁₂₁₋₁₃₆, and MAGE-A3₁₄₃₋₁₆₀ were dissolved in DMSO in equimolar amounts (peptide pool I) while the IL13Rα2 peptides IL13Rα2₃₄₁₋₃₅₅ and IL-13Rα2₃₅₁₋₃₆₅ were similarly dissolved together (peptide pool II) and used to stimulate freshly isolated PBMCs from 3 patients with primary GBMs. Significant (p<0.05) responses to both peptide pools were detected in all patients. Mean+SD are presented.

### Example 5

### T Cell Cytokine Profiles to Each Peptide Among Each Cohort

FIG. 3 shows T cell cytokine profiles to each peptide among each cohort. Each symbol represents the IFN-γ and IL-5 cytokine levels for a positive T cell response, defined as greater than 50 pg/ml and two standard deviations above the mean cytokine levels secreted after stimulation of cells with negative control MBP peptide for each subject. The mean cut-off for a positive cytokine response based on cytokine induced by stimulation with control MBP peptide was 895 pg/ml (range: 13-1298) and −314 pg/ml (range: 72-852) for IFN-γ and IL-5 among healthy subjects, and was 123 pg/ml (range: 0-286) and 312 pg/ml (range: 59-1347) for IFN-γ and IL-5 among GBM patients.

### Example 6

### Th1/2 Ratios of T Cell Responses to Each Peptide Among Each Cohort

FIG. 4 illustrates the Th1/2 ratios of T cell responses to each peptide among each cohort. The ratio of IFN-γ to IL-5 for each primary T cell response presented in FIG. 2 is presented. Patients with primary GBMs had significantly lower ratios compared to healthy subjects for every antigen examined. Patients with recurrent GBMs had significantly lower ratios compared to patients with primary GBMs in response to the MAGE-A3₁₄₃₋₁₆₀ and IL-13Rα2₃₅₁₋₃₆₅ epitopes (p<0.05).

In Table 10 is depicted the frequencies of response among subjects to the candidate glioma-associated T helper cell epitopes. Cytokine production was quantitated by ELISA, defining a positive T cell response for each patient as the amounts of IFN-γ or IL-5 that were >50 pg/mL and two standard deviations above the mean cytokine levels secreted after stimulation of cells from that patient with negative control MBP peptide. A total of 6 primary T cell responses were measured for each subject against each peptide. The chart summarizes responses as follows: blank cell=no response, +/++ cell=1-2 positive wells, +++/++++ cell=3-4 positive wells, and +++++/++++++ cell=5-6 positive wells. (+) symbols indicate the precise number of positive wells among six for each peptide.

TABLE 10

| | MAGE- | MAGE- | MAGE- | IL- | IL- |
|---|---|---|---|---|---|
| | A3₁₁₂₋₁₂₇ | A3₁₂₁₋₁₃₆ | A3₁₄₃₋₁₆₀ | 13Ra2₃₄₁₋₃₅₅ | 13Ra2₃₅₁₋₃₆₅ |
| Primary GBM 1 | | ++ | ++++ | + | +++ |
| Primary GBM 2 | ++ | ++ | +++ | ++ | ++ |
| Primary GBM 3 | ++ | ++++ | + | +++++ | ++ |
| Primary GBM 4 | + | ++ | + | | +++ |
| Primary GBM 5 | +++++ | ++++ | + | ++ | + |
| Primary GBM 6 | ++ | ++ | + | ++ | |
| Primary GBM 7 | +++ | ++++ | ++ | +++ | +++ |
| Primary GBM 8 | ++++ | ++ | +++++ | ++++ | +++++ |
| Recurrent GBM 1 | + | ++ | + | ++ | ++ |
| Recurrent GBM 2 | + | | | | |
| Recurrent GBM 3 | ++ | + | + | | + |
| Recurrent GBM 4 | ++ | + | + | | |
| Recurrent GBM 5 | + | + | | | ++ |
| Meningioma 1 | | + | | | |
| Meningioma 2 | | +++ | | | |
| Meningioma 3 | | | | | + |
| Meningioma 4 | | | + | | |
| Meningioma 5 | +++ | | | ++ | ++ |
| Meningioma 6 | + | + | + | + | ++++++ |
| Meningioma 7 | | | + | | |
| Healthy Subject 1 | +++ | +++ | +++ | | + |
| Healthy Subject 2 | | + | +++ | + | |
| Healthy Subject 3 | ++ | +++ | + | | |
| Healthy Subject 4 | ++ | + | +++++ | + | +++++ |
| Healthy Subject 5 | + | + | ++ | ++++ | ++ |
| Healthy Subject 6 | | + | + | + | ++ |

### Other Embodiments

Other embodiments of the invention will be apparent to those skilled in the art from a consideration of the specification or practice of the invention disclosed herein. It is intended that the specification and examples be considered as exemplary only, with the true scope of the invention being indicated by the following claims. The contents of any reference that is referred to herein are hereby incorporated by reference in their entirety.

As utilized herein, the terms “approximately,” “about,” “substantially”, and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the invention as recited in the appended claims.

It is also important to note that the construction and arrangement of the system, methods, and devices as shown in the various examples of embodiments is illustrative only. Although only a few embodiments have been described in detail in this disclosure, those skilled in the art who review this disclosure will readily appreciate that many modifications are possible (e.g., variations in sizes, dimensions, and proportions of the various elements, values of parameters, arrangements, use of materials, orientations, etc.) without materially departing from the novel teachings and advantages of the subject matter recited. For example, the order or sequence of any process or method steps may be varied or re-sequenced according to alternative embodiments. Other substitutions, modifications, changes and omissions may be made in the design, operating conditions and arrangement of the various examples of embodiments without departing from the spirit or scope of the present inventions.

While this invention has been described in conjunction with the examples of embodiments outlined above, various alternatives, modifications, variations, improvements and/or substantial equivalents, whether known or that are or may be presently foreseen, may become apparent to those having at least ordinary skill in the art. Accordingly, the examples of embodiments of the invention, as set forth above, are intended to be illustrative, not limiting. Various changes may be made without departing from the spirit or scope of the invention. Therefore, the invention is intended to embrace all known or earlier developed alternatives, modifications, variations, improvements and/or substantial equivalents.

The technical effects and technical problems in the specification are exemplary and are not limiting. It should be noted that the embodiments described in the specification may have other technical effects and can solve other technical problems.

1. An immunogenic composition comprising:
One or more peptides comprising a region having at least 75% sequence identity with 15-49 contiguous amino acids of SEQ ID NO. 1, wherein the one or more peptides comprises 49 or fewer contiguous amino acids from positions 112-160 of MAGE A3 protein.

2. The composition of claim 1, wherein the one or more peptides comprise a region having at least 80% sequence identity with 15-49 contiguous amino acids of SEQ ID NO. 1.

3. The composition of claim 1, wherein the one or more peptides comprise a region having at least 85% homology with 15-49 contiguous amino acids of SEQ ID NO. 1.

4. The composition of claim 1, wherein the one or more peptides comprise a region having at least 90% homology with 15-49 contiguous amino acids of SEQ ID NO. 1.

5. The composition of claim 1, wherein the one or more peptides comprise a region having at least 95% homology with 15-49 contiguous amino acids of SEQ ID NO. 1.

6. The composition of claim 1, wherein the one or more peptides comprise at least 15 contiguous amino acids of SEQ ID NO. 1.

7. An immunogenic composition comprising:
one or more peptides comprising a region having at least 75% sequence identity with at least 16-25 contiguous amino acids of SEQ ID NO. 2, wherein the one or more peptides comprises 25 or fewer contiguous amino acids from positions 341-365 of IL-13Rα2 protein.

8. The composition of claim 7, wherein the one or more peptides comprise a region having at least 80% sequence identity with 16-25 contiguous amino acids of SEQ ID NO. 2.

9. The composition of claim 7, wherein the one or more peptides comprise a region having at least 85% sequence identity with 16-25 contiguous amino acids of SEQ ID NO. 2.

10. The composition of claim 7, wherein the one of more peptides comprise a region having at least 90% sequence identity with 16-25 contiguous amino acids of SEQ ID NO. 2.

11. The composition of claim 7, wherein the one or more peptides comprise a region having at least 95% sequence identity with 16-25 contiguous amino acids of SEQ ID NO. 2.

12. The composition of claim 7, wherein the one or more peptides comprise at least 16 contiguous amino acids of SEQ ID NO. 2.

13. An immunogenic composition comprising:
One or more peptides comprising a region having at least 75% sequence identity with 15-49 contiguous amino acids of SEQ ID NO. 1, wherein the one or more peptides comprises 49 or fewer contiguous amino acids from positions 112-160 of MAGE A3 protein; and
one or more peptides comprising a region having at least 75% sequence identity with at least 16-25 contiguous amino acids of SEQ ID NO. 2, wherein the one or more peptides comprises 25 or fewer contiguous amino acids from positions 341-365 of IL-13Rα2 protein.

14. The composition of claim 13, wherein the one or more peptides comprise a region having at least 80% sequence identity with 15-49 contiguous amino acids of SEQ ID NO. 1 and wherein the one or more peptides comprise a region having at least 80% sequence identity with 16-25 contiguous amino acids of SEQ ID NO. 2.

15. The composition of claim 13, wherein the one or more peptides comprise a region having at least 85% sequence identity with 15-49 contiguous amino acids of SEQ ID NO. 1 and wherein the one or more peptides comprise a region having at least 85% sequence identity with 16-25 contiguous amino acids of SEQ ID NO. 2.

16. The composition of claim 13, wherein the one or more peptides comprise a region having at least 90% sequence identity with 15-49 contiguous amino acids of SEQ ID NO. 1 and wherein the one or more peptides comprise a region having at least 90% sequence identity with 16-25 contiguous amino acids of SEQ ID NO. 2.

17. The composition of claim 13, wherein the one or more peptides comprise a region having at least 95% sequence identity with 15-49 contiguous amino acids of SEQ ID NO. 1 and wherein the one or more peptides comprise a region having at least 95% sequence identity with 16-25 contiguous amino acids of SEQ ID NO. 2.

18. The composition of claim 13, wherein the one or more peptides comprise at least 15 contiguous amino acids of SEQ ID NO. 1 and wherein the one or more peptides comprise at least 16 contiguous amino acids of SEQ ID NO. 2.

19. The composition of claim 1, wherein each peptide is immunogenic.

20. The composition of claim 1, wherein the composition further comprises an adjuvant.

21. The composition of claim 20, wherein the adjuvant comprises alum.

22. The composition of claim 20, wherein the adjuvant comprises an immunologically active saponin fraction having adjuvant activity derived from the bark of the South American tree Quillaja Saponaria Molina.

23. The composition of claim 20, wherein the adjuvant comprises a TLR-3 agonist.

24. The composition of claim 23, wherein the adjuvant comprises polyriboinosinic:polyribocytidylic acid.

25. The composition of claim 20, wherein the adjuvant comprises a TLR-4 agonist.

26. The composition of claim 25, wherein the adjuvant comprises monophosphoryl lipid A or 3-deacyl monophosphoryl lipid A.

27. The composition of claim 20, wherein the adjuvant comprises a TLR-7/8 agonist.

28. The composition of claim 27, wherein the adjuvant comprises 1-isobutyl-1H-imidazo[4,5-c]quinolin-4-amine.

29. The composition of claim 20, wherein the adjuvant comprises a TLR-9 agonist.

30. The composition of claim 29, wherein the adjuvant comprises synthetic immunomodulatory oligonucleotide IMO-2055.

31. The composition of claim 20, wherein the adjuvant comprises a lipid vesicle

32. A method of treating a subject having malignant glioma comprising administering to the subject the composition of claim 1.

33. The method of claim 32, wherein the subject is an animal.

34. The method of claim 33, wherein the animal is a dog.

35. A method of treating a subject having melanoma comprising administering to the subject the composition of claim 1.

36. A method of immunizing a subject comprising administering to the subject the composition of claim 1.

37. The method of claim 32, wherein the composition is administered by intramuscular injection.

38. A method of measuring the immune response of a subject having malignant glioma prior to administering to the subject the composition of claim 1, by quantifying amounts of cytokine induced after exposure of peripheral blood mononuclear cells in vitro to the composition of claim 1.

39. A method of measuring the immune response of a subject having malignant glioma after administering to the subject the composition of claim 1, by quantifying amounts of cytokine induced after exposure of peripheral blood mononuclear cells in vitro to the composition of claim 1.

40. The method of claim 38 wherein the subject is an animal.

41. The method of claim 38 wherein the subject is a dog.

42. A method of measuring the immune response of a subject having melanoma prior to administering to the subject the composition of claim 1, by quantifying amounts of cytokine induced after exposure of peripheral blood mononuclear cells in vitro to the composition of claim 1.

43. A method of measuring the immune response of a subject having melanoma after administering to the subject the composition of claim 1, by quantifying amounts of cytokine induced after exposure of peripheral blood mononuclear cells in vitro to the composition of claim 1.


SEQUENCE LISTING (11 sequences)

SEQ ID NO: 1 - protein, 49 aa, Homo sapiens
KVAELVHFLLLKYRAREPVTKAEMLGSVVGNWQYFFPVIFSKASSSLQL

SEQ ID NO: 2 - protein, 25 aa, Homo sapiens
LLRFWLPFGFILILVIFVTQLLLRK

SEQ ID NO: 3 - protein, 16 aa, Artificial Sequence
  Description of Artificial Sequence Synthetic peptide
KVDELAHFLLRKYRAK

SEQ ID NO: 4 - protein, 16 aa, Artificial Sequence
  Description of Artificial Sequence Synthetic peptide
LRKYRAKELVTKAEML

SEQ ID NO: 5 - protein, 18 aa, Artificial Sequence
  Description of Artificial Sequence Synthetic peptide
WQYFFPVIFSKASSSLQL

SEQ ID NO: 6 - protein, 15 aa, Artificial Sequence
  Description of Artificial Sequence Synthetic peptide
LLRFWLPFGFILILV

SEQ ID NO: 7 - protein, 15 aa, Artificial Sequence
  Description of Artificial Sequence Synthetic peptide
ILILVIFVTQLLLRK

SEQ ID NO: 8 - protein, 15 aa, Artificial Sequence
  Description of Artificial Sequence Synthetic peptide
ILILVIFVTGLLLRK

SEQ ID NO: 9 - protein, 15 aa, Artificial Sequence
  Description of Artificial Sequence Synthetic peptide
ENPVVHFFKNIVTPR

SEQ ID NO: 10 - protein, 16 aa, Artificial Sequence
  Description of Artificial Sequence Synthetic peptide
FLLLKYRAREPVTKAE

SEQ ID NO: 11 - protein, 15 aa, Artificial Sequence
  Description of Artificial Sequence Synthetic peptide
FFPVIFSKASSSLQL